(19) United States
(12) Patent Application Publication
(10) Pub. No.: US 2026/0295032 A1
(43) Pub. Date: Oct. 1, 2026

(54) METHOD FOR TARGETING LIPID NANOVESICLES TO SPLEEN AND COMPOSITION THEREFOR

(71) Applicants: KAWASAKI INSTITUTE OF INDUSTRIAL PROMOTION, Kawasaki-shi (JP); NANO MRNA Co., Ltd., Tokyo (JP)

(72) Inventors: Satoshi UCHIDA, Kawasaki-shi (JP); Miki MASAI, Tokyo (JP); Anjaneyulu DIRISALA, Kawasaki-shi (JP); Kazunori KATAOKA, Tokyo (JP)

(21) Appl. No.: 19/478,458

(22) PCT Filed: Apr. 25, 2024
(86) PCT No.: PCT/JP2024/016221

§ 371 Date: Oct. 24, 2025

(30) Foreign Application Priority Data
Apr. 26, 2023 (JP) 2023-072199

(51) Int. Cl. A61K 39/215; A61K 9/51; A61K 39/00; A61K 47/34; A61P 37/04

(52) U.S. Cl. CPC A61K 39/215; A61K 9/5123; A61K 47/34; A61P 37/04; A61K 2039/53

(57) ABSTRACT

The present disclosure relates to a method of delivering a molecule of interest to the spleen. The present disclosure also relates to a method of delivering lipid nanoparticles to the spleen. The present disclosure further relates to a method of inducing antigen-specific immunity in a subject. The present disclosure comprises using a cationic polymer simultaneously with or before the administration of a lipid nanovesicle encapsulating the molecule of interest.

### TECHNICAL FIELD

The present disclosure relates to a method of targeting lipid nanovesicles to the spleen and compositions therefor.

### BACKGROUND ART

Lipid nanoparticles (LNP) are widely used as a technology for intracellular delivery of mRNA, for example, in coronavirus vaccines (Non-patent Literature 1). The lipid nanoparticles used here include cationic/ionizable lipids, non-cationic lipids, cholesterol, and PEGylated lipids (see Patent Literatures 1 and 2). The cationic/ionizable lipids enable complex formation with mRNA, and the PEGylated lipids (or PEG lipids) are thought to be involved in controlling the particle size of the lipid nanoparticles formed by microfluidics. Cell membranes contain phospholipids; therefore, including phospholipids in lipid nanoparticles enhances affinity with cells, serving to facilitate cellular uptake after contact with the cell. Cholesterol stabilizes the structure of the lipid nanoparticles. mRNA, including modified nucleotides, is encapsulated in the lipid nanoparticles. Specifically, in the encapsulated mRNA, uridine is replaced with pseudouridine (see Patent Literature 3).

Lipid nanoparticles are used in ONPATTRO (trademark) as a technology for delivering nucleic acids to the liver (Non-patent Literature 2). ONPATTRO is a nucleic acid drug for transthyretin-type familial amyloid polyneuropathy, and its active ingredient is siRNA that specifically inhibits the production of transthyretin (TTR). ONPATTRO contains lipid nanoparticles comprising siRNA against TTR, and is administered intravenously by drip infusion. The administered lipid nanoparticles are taken up by the liver, where they can suppress TTR expression in hepatocytes and improve the symptoms of transthyretin-type familial amyloid polyneuropathy. Thus, lipid nanovesicles are known to be effective as a means of delivering encapsulated molecules to the liver.

On the other hand, there are reports that nucleic acid delivery technology using lipid nanoparticles induces autoimmune diseases (particularly autoimmune hepatitis) (Non-patent Literature 3). There are also reports that nucleic acid delivery technology using lipid nanoparticles causes myocarditis (Non-patent Literature 4).

It has been shown that polyion complex micelles improve blood retention when co-administered with a cationic polymer (Non-patent Literature 5 and Patent Literature 4). Non-patent Literature 5 and Patent Literature 4 disclose that adeno-associated virus 8 (AAV8) increases accumulation in the heart and skeletal muscle when co-administered with a cationic polymer.

### PRIOR ART DOCUMENTS

### Patent Literatures

[Patent Literature 1] U.S. Pat. No. 8,058,069B2
[Patent Literature 2] U.S. Pat. No. 8,278,036B
[Patent Literature 3] U.S. Pat. No. 8,278,036B
[Patent Literature 4] US2021/0038634A

### Non-Patent Literatures

[Non-patent Literature 1] Linde Schoenmaker, et al., International Journal of Pharmaceutics, 601:120586, 2021.
[Non-patent Literature 2] https://www.onpattro.com/
[Non-patent Literature 3] Tobias Boettler et al., Journal of Hepatology, 7:653-659, 2022
[Non-patent Literature 4] Lael Yonker et al., Circulation, 147:867-876, 2023
[Non-patent Literature 5] Anjaneyulu Dirasa et al., Science Advances 6, eabb8133, (2020)

### SUMMARY OF INVENTION

The present disclosure provides a method of targeting lipid nanovesicles to a specific organ and compositions therefor. The present disclosure also provides a method of targeting lipid nanovesicles to the spleen and compositions therefor.

According to the present disclosure, lipid nanovesicles mainly accumulate in and are taken up by the liver after intravenous administration. In contrast, according to the present disclosure, by administering lipid nanovesicles in combination with a cationic polymer, the nanovesicles are modified to prevent accumulation in the liver and selectively accumulate in other specific organs (particularly the spleen).

According to the present disclosure, for example, the following inventions are provided:
(1) A method of delivering a molecule of interest to the spleen of a subject in need thereof, comprising administering to the subject a cationic polymer (particularly, a free cationic polymer) and a lipid nanoparticle (LNP) encapsulating the molecule of interest, wherein at least a portion of the cationic polymer is administered simultaneously with the LNP administration or before the LNP administration, and preferably, thereby delivering more molecules of interest to secondary lymphoid tissues (e.g., the spleen) compared to administering the same amount of the LNP without administering said portion of the cationic polymer.
(2) The method according to (1), wherein the cationic polymer is a block copolymer with a non-charged hydrophilic polymer.
(3) The method according to (1) or (2), wherein the LNP comprises (i) a cationic lipid or ionizable lipid, (ii) a neutral lipid, (iii) cholesterol, and (iv) a PEG lipid {where, for example, it may comprise 40 to 60 mol % cationic lipid, 4 to 15 mol % neutral lipid, 30 to 45 mol % cholesterol, and 0.5 to 2 mol % PEG lipid}.
(4) The method according to any one of (1) to (3), wherein the molecule of interest is a nucleic acid.
(5) The method according to any one of (1) to (4), wherein the molecule of interest is a messenger RNA (mRNA).
(6) The method according to (5), wherein the molecule of interest is an mRNA encoding an antigen protein, thereby causing the antigen protein to be expressed in the spleen of the subject and inducing antigen-specific immunity in the subject.
(7) The method according to (6), wherein the antigen protein is the spike protein of SARS-CoV-2.
(8) A composition comprising a cationic polymer for use in the method according to any one of (1) to (7).
(9) The composition according to (8), further for use in suppressing the distribution of the molecule of interest to the liver (and preferably, thereby preventing the induction of immune tolerance to the antigen).
(10) The composition according to (9), which is a pharmaceutical composition.
(21) The method or composition according to any of the above, wherein the lipid nanoparticle comprises an ionizable lipid (preferably, comprises an ionizable lipid, a neutral lipid, sterols, and a PEG lipid), and encapsulates a nucleic acid (particularly mRNA).
(22) The method or composition according to the above (particularly, the method or composition according to (21)), wherein the lipid nanoparticle is one prepared by a microfluidic device.
(23) The method or composition according to (21) or (22), wherein the lipid nanoparticle is positively charged.
(24) The method or composition according to any of the above (particularly, the method or composition according to (21) or (22)), wherein the mRNA comprises pseudouridine instead of uridine.
(25) The method or composition according to any of the above (particularly, the method or composition according to any one of (21) to (24)), wherein the cationic polymer is polylysine or polyornithine having branched PEG.
(26) The method or composition according to any of the above (particularly, the method or composition according to any one of (21) to (25)), wherein the cationic polymer is polylysine or polyornithine having branched PEG, the PEG portion is a branched PEG having two PEG chains with an average molecular weight of 20 to 50 kD, and the polylysine or polyornithine has an average degree of polymerization of 10 to 70, 15 to 60, 10 to 40, or 10 to 30 (e.g., about 20).
(41) The method or composition according to any of the above, wherein the cationic polymer is administered before the lipid nanoparticle.
(42) The method or composition according to any of the above, wherein the cationic polymer is administered while the lipid nanoparticle is being administered.
(43) The method or composition according to any of the above, wherein the cationic polymer is administered after the lipid nanoparticle.
(61) The method or composition according to any of the above, wherein the mRNA encodes a protein that is an immunogen and induces immunogen-specific immunity in the subject.
(62) The method or composition according to (61), wherein the immunogen comprises one or more selected from the group consisting of a viral antigen, a bacterial antigen, a fungal antigen, a parasite antigen, and a tumor antigen.
(63) The method or composition according to (61), wherein the immunogen is a viral antigen.
(64) The method or composition according to (61), wherein the immunogen is the S protein of β coronavirus.
(65) The method or composition according to (61), wherein the immunogen is the S protein of SARS-COV or SARS-COV-2.
(66) The method or composition according to (61), wherein the immunogen is a tumor antigen.
(81) The method or composition according to any of the above, wherein the mRNA has one or more target sequences for microRNA that is expressed tissue-specifically in the UTR.
(82) The method or composition according to any of the above, wherein the mRNA has one or more target sequences for microRNA that is expressed liver-specifically in the UTR.
(83) The method or composition according to any of the above, wherein the mRNA has one or more target sequences for miR-122 or miR-192 in the UTR.
(84) The method or composition according to any one of (81) to (83), wherein the UTR is the 3′ UTR.
(85) The method or composition according to any one of (81) to (84), wherein the amount of protein expression from the mRNA in the liver is reduced compared to a case where the mRNA does not have the target sequence in the UTR.
(101) The method or composition according to any of the above, wherein the cationic polymer and LNP are administered intravenously.
(102) The method or composition according to any of the above, wherein the cationic polymer is administered intravenously and the LNP is administered intramuscularly.
(103) The method or composition according to any of the above, wherein the cationic polymer is administered intravenously and the LNP is administered intradermally or subcutaneously.
(104) The method or composition according to any of the above, wherein the cationic polymer and LNP are each administered intravenously, intramuscularly, or intradermally or subcutaneously.
(105) The method or composition according to any of the above, wherein the LNP is administered intravenously.
(106) The method or composition according to any of the above, wherein the LNP is administered intramuscularly.
(107) The method or composition according to any of the above, wherein the LNP is administered intradermally or subcutaneously.
(108) The method or composition according to (105), wherein the cationic polymer is administered intravenously.
(109) The method or composition according to (105), wherein the cationic polymer is administered intramuscularly.
(110) The method or composition according to (105), wherein the cationic polymer is administered intradermally or subcutaneously.
(111) The method or composition according to (106), wherein the cationic polymer is administered intravenously.
(112) The method or composition according to (106), wherein the cationic polymer is administered intramuscularly.
(113) The method or composition according to (106), wherein the cationic polymer is administered intradermally or subcutaneously.
(114) The method or composition according to (107), wherein the cationic polymer is administered intravenously.
(115) The method or composition according to (107), wherein the cationic polymer is administered intramuscularly.
(116) The method or composition according to (107), wherein the cationic polymer is administered intradermally or subcutaneously.
(121) The composition according to any of the above, comprising the cationic polymer.
(122) The composition according to any of the above, comprising the LNP.
(123) The composition according to any of the above, comprising the cationic polymer and the LNP.
(124) The composition according to any of the above (particularly, the composition according to (122) or (123)), wherein the LNP encapsulates mRNA encoding an antigen and is for use in inducing specific immunity to the antigen.
(125) The composition according to any of the above (particularly, the composition according to (124)), wherein the antigen comprises any one or more selected from the group consisting of a viral antigen, a bacterial antigen, a fungal antigen, a parasite antigen, and a tumor antigen.

### BRIEF DESCRIPTION OF THE DRAWINGS

FIG. 1 shows an example of the administration scheme of the cationic polymer and lipid nanoparticles (LNP) of the present disclosure. In the figure, polylysine having two polyethylene glycols at one end is shown as the cationic polymer.

FIG. 2A shows an example of the structure of the mRNA of the present disclosure. In the figure, the mRNA has a cap structure and a poly A sequence to express the protein encoded in the coding region. The mRNA also has three miR-122 target sequences.

FIG. 2B shows the luciferase expression level in each tissue of mice administered with LNP encapsulating mRNA having the structure shown in FIG. 2A, wherein the coding region encodes luciferase, according to the scheme shown in FIG. 1 {however, the LNP is administered intravenously}.

FIG. 2C shows the distribution of luciferase expression in mice administered with LNP encapsulating mRNA having the structure shown in FIG. 2A, wherein the coding region encodes luciferase, according to the scheme shown in FIG. 1 {however, the LNP is administered intravenously}. Coating (+) and (−) in the figure indicate the presence or absence of pre-administration of the cationic polymer, respectively (the same applies hereinafter). miR (+) and (−) indicate the presence or absence of the miR-122 target sequence in the administered mRNA, respectively (the same applies hereinafter).

FIG. 3 shows the presence of antigen-specific immune cells in splenocytes of mice administered with LNP encapsulating mRNA having the structure shown in FIG. 2A, wherein the coding region encodes ovalbumin (OVA), according to the scheme shown in FIG. 1 {however, the LNP is administered intravenously}.

FIG. 4A shows the luciferase expression level in each tissue of mice administered with LNP encapsulating mRNA having the structure shown in FIG. 2A, wherein the coding region encodes luciferase, according to the scheme shown in FIG. 1 {however, the LNP is administered intramuscularly}.

FIG. 4B shows the distribution of luciferase expression in mice administered with LNP encapsulating mRNA having the structure shown in FIG. 2A, wherein the coding region encodes luciferase, according to the scheme shown in FIG. 1 {however, the LNP is administered intramuscularly}.

FIG. 5 shows antibody production and the presence of antigen-specific immune cells in splenocytes in mice administered with LNP encapsulating mRNA whose coding region encodes the S protein of SARS-COV-2 Wuhan strain, according to the scheme shown in FIG. 1 {however, the LNP is administered intramuscularly}.

FIG. 6 shows the luciferase expression level in each tissue of mice administered with lipid nanoparticles consisting of D-Lin-MC3-DMA, DSPC, PEG2000-DMG, and cholesterol, encapsulating mRNA encoding luciferase, according to the scheme shown in FIG. 1 {however, the LNP is administered intravenously}.

FIG. 7 shows the in vivo kinetics of intravenously administered labeled cationic polymers (homopolylysine, PEGylated polylysine, branched PEGylated polylysine).

FIG. 8 shows luciferase expression in each tissue and the presence of OVA-specific immune cells in splenocytes of C57BL6J mice administered with LNP encapsulating mRNA whose coding region encodes luciferase or OVA, according to the scheme shown in FIG. 1 {however, the LNP is administered intravenously}.

FIG. 9 shows the distribution of luciferase expression in C57BL6J strain tumor cell graft mice administered with LNP encapsulating mRNA whose coding region encodes luciferase, according to the scheme shown in FIG. 1 {however, the LNP is administered intratumorally}.

FIG. 10 shows the luciferase expression level in the liver and spleen of cationic polymer pre-administration groups and non-administration groups, where cationic lipoplex and anionic lipoplex containing mRNA encoding luciferase were administered, respectively.

### MODE FOR CARRYING OUT THE INVENTION

### Definition of Terms

In the present specification, defined terms have the meaning as defined. Undefined terms have the meaning generally used in the technical field. The singular form does not exclude the plural. “Comprising” means that elements other than the specified ones may be included, and “consisting of” means that elements other than the specified ones are substantially not included (e.g., only unavoidable contamination of said elements during manufacturing, or only an amount that does not substantially affect the function, or only an amount below the detection limit), or not included.

In the present specification, a “subject” can be an animal. Examples of animals include vertebrates, such as mammals and birds. Examples of mammals include primates such as humans, rodents such as mice and rats, and domestic mammals such as cattle, horses, sheep, donkeys, sheep, goats, llamas, and camels. Examples of birds include chickens.

In the present specification, a “molecule of interest” refers to a molecule that is beneficial to deliver to a target tissue, such as a nucleic acid and a small molecule compound.

In the present specification, a “target tissue” is an organ or tissue in a body of a subject. The target tissue can be the spleen.

In the present specification, a “nucleic acid” includes deoxyribonucleic acid (DNA), and ribonucleic acid (RNA), as well as modified nucleic acids thereof. Examples of nucleic acids include, but are not limited to, nucleic acids comprising only DNA, nucleic acids comprising only RNA, nucleic acids comprising only DNA and RNA, nucleic acids comprising DNA and modified nucleic acids, nucleic acids comprising RNA and modified nucleic acids, and nucleic acids comprising DNA, RNA, and modified nucleic acids.

In the present specification, “messenger RNA” (mRNA) is an RNA having a region that encodes a protein (open reading frame) and that can produce a protein as a translation product in a cell. mRNA usually has a 5′ cap structure, a 5′ untranslated region (5′ UTR), a coding region, a 3′ UTR, and a polyadenine (poly A) sequence. The 5′ cap structure can be a cap comprising, for example, N7-methylguanosine (m7G) (e.g., m7GpppG cap, 3′-O-methyl-m7GpppG cap). The 5′ UTR and 3′ UTR, for example, promote the translation of the protein from the mRNA. The mRNA is suitable or has a structure suitable for translating the encoded protein.

In the present specification, a “modified nucleic acid” is a derivative of DNA or RNA and can be DNA or RNA modified for purposes such as enhancement of hybridization ability, stability against degradation, and thermal stability. Examples of modified nucleic acids include, but are not limited to, nucleic acids modified with a fluorescent dye, biotinylated nucleic acids, and nucleic acids into which a cholesteryl group has been introduced. RNA may be modified with 2′-O-methyl modification or 2′-fluoro modification or 2′-methoxyethyl (MOE) modification on the base to enhance stability, or the phosphodiester bond of the nucleic acid backbone may be replaced with a phosphorothioate bond. Modified nucleic acids include cross-linked nucleic acids. Examples of such artificial nucleic acids include locked nucleic acid (LNA), which is a cross-linked DNA in which the oxygen atom at the 2′ position and the carbon atom at the 4′ position are cross-linked via methylene; ENA, in which the oxygen atom at the 2′ position and the carbon atom at the 4′ position are cross-linked via ethylene; BNACOC in which the oxygen atom at the 2′ position and the carbon atom at the 4′ position are cross-linked via —CH₂OCH₂—; BNANC, in which the oxygen atom at the 2′ position and the carbon atom at the 4′ position are cross-linked via —NR—CH₂— {where R is a methyl or hydrogen atom}; cMOE, in which the oxygen atom at the 2′ position and the carbon atom at the 4′ position are cross-linked via —CH₂(OCH₃)—; cEt, in which the oxygen atom at the 2′ position and the carbon atom at the 4′ position are cross-linked via —CH₂(CH₃)—; AmNA, in which the carbon atoms at the 2′ position and the 4′ position are cross-linked via an amide; scpBNA, in which the oxygen atom at the 2′ position and the carbon atom at the 4′ position are cross-linked via methylene and a cyclopropane is formed at the 6′ position; peptide nucleic acid (PNA), whose main chain is a polymer in which N-(2-aminoethyl)glycine is amidated instead of deoxyribose or ribose; and morpholino oligos, in which the bases are linked by a morpholine ring (e.g., U.S. Pat. No. 9,469,664B, which is incorporated herein by reference in its entirety). The modified nucleic acid may have an acidic property, but does not need to be acidic. Examples of modified mRNA include mRNA containing modified nucleosides. Examples of mRNA containing modified nucleosides include modified nucleosides described in U.S. Pat. No. 8,278,036B, which is incorporated herein by reference in its entirety. Modified nucleosides include, for example, pseudouridine, which is known as a modified nucleoside for in vivo expression of mRNA. A modified nucleoside can substitute for an unmodified nucleoside. Pseudouridines include, for example, 1-methyl-3-(amino-5-carboxypropyl) pseudouridine (m¹acp³Ψ), 1-methylpseudouridine (m1Ψ), 2′-O-methylpseudouridine (Ψm), 5-methyldihydrouridine (m5D), 3-methylpseudouridine (m3′), and the like, which can substitute for uridine. The modified mRNA preferably comprises pseudouridine (more preferably m1Ψ), and may further comprise 5-methylcytidine.

In the present specification, a “lipid nanoparticle” (LNP) means a nanovesicle composed of lipid molecules. Examples of lipid nanoparticles include the nucleic acid-lipid particles described in U.S. Pat. No. 8,058,069B, which is incorporated herein by reference in its entirety. Amphiphilic lipids can form lipid nanoparticles in an aqueous solution. A person skilled in the art can appropriately select a lipid and form a lipid nanoparticle from the lipid.

In the present specification, the “spike protein” can be the spike protein of β coronavirus, for example, the protein encoded at positions 21563 to 25384 of the SARS-CoV-2 genome registered with the National Center for Biotechnology Information (NCBI) under GenBank accession number: MN908947.3. The SARS-COV-2 spike protein has the amino acid sequence registered with NCBI under GenBank accession number: QHD43416.1. The spike protein includes S1 and S2. S1 is present at positions 13 to 541 of the above amino acid sequence, and S2 is present at positions 543 to 1208 of the above amino acid sequence. S1 further has an N-terminal domain (NTD) and a receptor-binding domain (RBD). NTD is present at positions 13 to 304 of the above amino acid sequence, and RBD is present at positions 319 to 541. S1 and S2 are cleaved inside the cell, produced as separate peptides, and form a complex during virus particle formation. The spike protein is also called the S protein. The spike protein forms a trimer, binds to angiotensin-converting enzyme 2 (ACE2) expressed on a host cell, and can infect the cell. Any spike protein of a natural virus (including a variant virus) (having an amino acid sequence corresponding to the amino acid sequence registered with NCBI under GenBank accession number: QHD43416.1) can be used as the spike protein. Examples of the amino acid sequence of a spike protein of a variant virus are as follows:
αstrain: has the mutations HV69-70del, Y144del, N501Y, A570D, D614G, P681H, T716I, S982A, and D1118H relative to the sequence of the wild-type strain.
β strain: has the mutations L18F, D80A, D215G, LAL242-244del, R246I, K417N, E484K, N501Y, D614G, and A701V relative to the sequence of the wild-type strain.
γ strain: has the mutations L18F, T20N, P26S, D138Y, R190S, K417T, E484K, N501Y, D614G, H655Y, T1027I, and V1176F.
δ strain: has the mutations T19R, G142D, EF156-157del, R158G, L452R, T478K, D614G, P681R, and D950N relative to the sequence of the wild-type strain.
strain: has the mutations A67V, HV69-70del, T95I, G142D, VYY143-145del, N211del, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F relative to the sequence of the wild-type strain.

Regarding SARS-COV-2 variants, as of February 2024, a global replacement is underway from sublineages of the XBB lineage (recombinant of BJ.1 lineage and BM.1.1.1 lineage), such as the EG.5.1 lineage, to the JN.1 lineage, a sublineage of the BA.2.86 lineage, and its sub-variants (WHO, 2024a, covSPECTRUM, 2024). Various variant strains, including the above lineages, sublineages, and sub-variants, have an S protein, and lipid nanoparticles containing mRNA encoding the S protein can be prepared as a vaccine.

### <Method of the Present Disclosure and Compositions for Use in the Method>

The present disclosure provides a method of administering a molecule of interest to a subject. In one preferred embodiment, the method of the present disclosure further comprises administering a cationic polymer before, during, or after administering the molecule of interest to the subject. The cationic polymer changes the in vivo kinetics of the molecule of interest. Thus, the subject can be a subject having an effective blood concentration of the molecule of interest. In one preferred embodiment, the method of the present disclosure further comprises administering a cationic polymer before administering the molecule of interest to the subject. In one preferred embodiment, the method of the present disclosure further comprises administering a cationic polymer to a subject having an effective blood concentration of the molecule of interest. In one preferred embodiment, the subject is a subject to whom the cationic polymer has been administered (in whom the clearance of the lipid nanoparticles by the liver will be reduced by the administration).

In one preferred embodiment, the molecule of interest is encapsulated in a lipid nanoparticle. The lipid nanoparticle is delivered to the spleen in the presence of the cationic polymer. The delivery can be selective to the spleen. Further, the lipid nanoparticle shows reduced accumulation in the liver and/or reduced clearance in the liver in the presence of the cationic polymer. In one preferred embodiment, the expression level in the spleen/expression level in the liver (spleen/liver ratio) may be 1 or more, 2 or more, 3 or more, 4 or more, 5 or more, 6 or more, 7 or more, 8 or more, 9 or more, or 10 or more, and may be 100 or less, 90 or less, 80 or less, 70 or less, 60 or less, 50 or less, 40 or less, 30 or less, or 20 or less. In one embodiment, it can be 1 to 20. In this embodiment, the cationic polymer can be free. A free cationic polymer means one that is not incorporated into the lipid nanoparticle.

A lipid nanoparticle may comprise one or more, or preferably all, selected from the group consisting of a cationic lipid, a non-cationic lipid (or neutral lipid), sterols, and a PEG lipid. In one embodiment, the cationic lipid is an ionizable cationic lipid. In one embodiment, the non-cationic lipid is a neutral lipid. In one embodiment, the sterols include cholesterol. Note that the cationic lipid is different from the cationic polymer.

In one embodiment, examples of the cationic lipid include 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl] heptadecan-8-amine (L530). In one embodiment, the cationic lipid may include L608, or be L608. In one embodiment, the cationic lipid may include L530, or be L530.

In one embodiment, the cationic lipid can be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 of US20130150625); 2-amino-3-[(9Z)-octadeca-9-en-1-yloxy]-2-{[(9Z)-octadeca-9-en-1-yloxy]methyl}propan-1-ol (Compound 2 of US20130150625); 2-amino-3-[(9Z,12Z)-octadeca-9,12-dien-1-yloxy]-2-[(octyloxy)methyl]propan-1-ol (Compound 3 of US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 of US20130150625); or any pharmaceutically acceptable salt or stereoisomer thereof.

In one embodiment, the ionizable cationic lipid (also called ionizable lipid) can be 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy) heptadecanedioate (L319).

In one embodiment, the neutral lipids include 1,2-distearoyl-sn-glycero-3-phosphocholine (DSPC), dipalmitoylphosphatidylcholine (DPPC), PO phosphatidylcholine (POPC), 1,2-dioleoyl-sn-glycero-3-phosphoethanolamine (DOPE), and sphingomyelin (SM). The neutral lipid can be neutral as a whole molecule under neutral pH conditions.

Sterols include, for example, animal-derived sterols such as cholesterol, cholesterol succinate, lanosterol, dihydrolanosterol, desmosterol, and dihydrocholesterol; plant-derived sterols (phytosterols) such as stigmasterol, sitosterol, campesterol, and brassicasterol; and microorganism-derived sterols such as thymosterol and ergosterol. The lipid nanoparticle preferably comprises a sterol, and more preferably comprises cholesterol.

The lipid nanoparticle may comprise a lipid having a polyalkylene glycol modification. Polyalkylene glycols include, for example, polyethylene glycol, polypropylene glycol, polytetramethylene glycol, and polyhexamethylene glycol. The weight average molecular weight of the polyalkylene glycol is, for example, about 300 to 10,000, preferably about 500 to 10,000, and more preferably about 1,000 to 5,000.

As the polyethylene glycol modification of the lipid, stearylated polyethylene glycol (e.g., PEG45 stearate (STR-PEG45)) can be used. Further, as the PEG lipid, polyethylene glycol derivatives such as N-[carbonyl-methoxypolyethylene glycol-2000]-1,2-dipalmitoyl-sn-glycero-3-phosphoethanolamine, N-[carbonyl-methoxypolyethylene glycol-5000]-1,2-dipalmitoyl-sn-glycero-3-phosphoethanolamine, N-[carbonyl-methoxypolyethylene glycol-750]-1,2-distearoyl-sn-glycero-3-phosphoethanolamine, N-[carbonyl-methoxypolyethylene glycol-2000]-1,2-distearoyl-sn-glycero-3-phosphoethanolamine, N-[carbonyl-methoxypolyethylene glycol-5000]-1,2-distearoyl-sn-glycero-3-phosphoethanolamine, and 1,2-dimyristoyl-rac-glycero-3-methoxypolyethylene glycol-2000 (PEG-DMG) can also be used.

The lipid nanoparticle preferably has an average particle size of 400 nm or less, and more preferably has an average particle size of 300 nm or less. Note that the average particle size of the lipid nanoparticle means the number average particle size measured by dynamic light scattering (DLS). Measurement by DLS can be performed by a conventional method using a commercially available DLS device or the like. The polydispersity index (PDI) of the lipid nanoparticle may be about 0.05 to 0.5, about 0.05 to 0.4, about 0.05 to 0.3, preferably about 0.05 to 0.2, more preferably about 0.05 to 0.15, or about 0.1. The lipid nanoparticle can be prepared using a microfluidic device according to a conventional method. It is also easy to control the PDI of the LNP to about 0.1 using a conventional method.

In one embodiment, the lipid nanoparticle may comprise about 20 mol % to about 60 mol % cationic lipid, about 0.5 mol % to about 15% PEG-modified lipid, about 25 mol % to about 55% sterol, and about 20 mol % to about 60 mol % non-cationic lipid. In one preferred embodiment, the lipid nanoparticle comprises about 45 mol % to about 50 mol % cationic lipid, about 9 to 10 mol % non-cationic lipid (preferably neutral lipid), about 35 mol % to about 45 mol % sterol (preferably cholesterol), and about 1.5 mol % to about 2 mol % PEG lipid.

The lipid nanoparticle may have a positive charge in an aqueous solution. The lipid nanoparticle may also have a negative charge in an aqueous solution. The lipid nanoparticle may also be non-charged in an aqueous solution. In one preferred embodiment, the lipid nanoparticle has a positive charge in an aqueous solution.

The molecule of interest can be mRNA. In the mRNA, for example, one or all uridines are substituted with modified uridine. Examples of modified uridine include pseudouridine, and preferably 1-methylpseudouridine (m1!′).

The cationic polymer can be a homopolymer or a copolymer. The copolymer can be, for example, a binary polymer or a terpolymer. The copolymer can be an alternating copolymer, a random copolymer, a block copolymer, or a graft copolymer. Cationic polymer or cationic polymer portion include, for example, cationic natural amino acids and cationic non-natural amino acids, for example, cationic natural amino acids such as histidine, tryptophan, ornithine, arginine, and lysine, and/or a cationic polymer or cationic polymer portion having a group represented by —(NH—(CH₂)₂)ₚ—NH₂ as a side chain {where p is an integer from 1 to 5}, for example, a cationic polymer or cationic polymer portion of a cationic non-natural amino acid having the above cationic side chain, for example, a cationic polymer or cationic polymer portion of a cationic non-natural amino acid such as aspartic acid or glutamic acid having the above cationic side chain. In one embodiment of the present invention, the polycation block is a cationic polymer or cationic polymer portion having a group represented by —(NH—(CH₂)₂)ₚ—NH₂ as a side chain {where p is an integer from 1 to 5}. Here, preferred examples of cationic natural amino acids include histidine, tryptophan, ornithine, arginine, and lysine; more preferably arginine, ornithine, and lysine; still more preferably ornithine and lysine; and even more preferably lysine. In one embodiment of the present invention, the cationic polymer or cationic polymer portion can be polylysine or polyornithine. In one embodiment of the present invention, 40%, 50%, 60%, 70%, 80%, 90%, 95%, 98%, or 100% of the monomer units in the polymer have a group represented by —(NH—(CH₂)₂)ₚ—NH₂ as a side chain {where p is an integer from 1 to 5}.

The cationic polymer can be in the form of a block copolymer with a non-charged hydrophilic polymer. The non-charged hydrophilic polymer block is a pharmaceutically acceptable polymer. Such polymers include, for example but are not limited to, polyalkylene glycol, poly(2-oxazoline), polysaccharide, polyvinyl alcohol, polyvinylpyrrolidone, polyacrylamide, polymethacrylamide, polyacrylate ester, polymethacrylate ester, and poly(2-methacryloyloxyethyl phosphorylcholine). As non-charged hydrophilic polymer, polyalkylene glycol and poly(2-oxazoline) are preferably used, and polyalkylene glycol may be particularly preferably used. Polyethylene glycol (PEG) may be preferably used as the polyalkylene glycol.

The average degree of polymerization of the cationic polymer can be 10 or more, 15 or more, 20 or more, 30 or more, or 40 or more (e.g., it can be 80 or less, 70 or less, 60 or less, or 50 or less). Note that in the present specification, the average degree of polymerization indicates the weight average degree of polymerization (Pw) or the number average degree of polymerization (Pn), and Pw and Pn are equivalent values (i.e., P w/P n=1 to 1.2 or 1 to 1.1).

In the copolymer comprising the cationic polymer portion and the PEG portion, the average molecular weight of the PEG portion can be, for example, 10 kD or more, 15 kD or more, 20 kD or more, 30 kD or more, or 40 kD or more (e.g., it can be 80 kD or less, 70 kD or less, 60 kD or less, or 50 kD or less), preferably 20 kD or more, and more preferably 30 kD or more. In the copolymer comprising the cationic polymer portion and the PEG portion, the cationic polymer may have an average degree of polymerization of 10 or more, 15 or more, 20 or more, 30 or more, or 40 or more (e.g., it can be 80 or less, 70 or less, 60 or less, or 50 or less). From the viewpoint of increasing the bulk of the PEG portion, the PEG portion can be a single-chain PEG with a large average molecular weight, for example, 40 kD or more, 50 kD or more, 60 kD or more, or 70 kD or more (e.g., it can be 80 kD or less, 70 kD or less, 60 kD or less, or 50 kD or less), or a branched PEG having multiple PEG chains of 10 kD or more, 15 kD or more, 20 kD or more, 30 kD or more, or 40 kD or more (e.g., it can be 80 kD or less, 70 kD or less, 60 kD or less, or 50 kD or less). From the viewpoint of increasing the bulk of the PEG portion, branched PEG may be preferably used. Note that in the present specification, the average molecular weight indicates the weight average molecular weight (Mw) or the number average molecular weight (Mn), and Pw and Pn are equivalent values (i.e., Mw/M n=1 to 1.2 or 1 to 1.1).

In one embodiment, the copolymer comprising the cationic polymer portion and the PEG portion has a PEG portion that is a branched PEG having multiple PEG chains of 10 kD or more, 15 kD or more, 20 kD or more, 30 kD or more, or 40 kD or more, and the cationic polymer portion may have an average degree of polymerization of 15 or more, 20 or more, 30 or more, or 40 or more. In this specific embodiment, the copolymer comprising the cationic polymer and PEG has a PEG portion that is a branched PEG having multiple PEG chains of 20 kD or more, 30 kD or more, or 40 kD or more, and the cationic polymer portion may have an average degree of polymerization of 20 or more, 30 or more, or 40 or more.

In one embodiment, the copolymer comprising the cationic polymer portion and the PEG portion has a PEG portion that is a single-chain PEG of 40 kD or more, and the cationic polymer portion may have an average degree of polymerization of 15 or more, 20 or more, 30 or more, 40 or more, 50 or more, 60 or more, or 70 or more (e.g., it can be 80 or less, 70 or less, 60 or less, or 50 or less. Particularly in the case of a single-chain PEG, the larger the average molecular weight, the greater the effect of improving blood retention).

In one specific embodiment, the average degree of polymerization of the cationic polymer block is 15 to 30, the cationic polymer block is linked to a branched PEG, and the total average molecular weight of the PEG portion of the branched PEG may be 40 kD to 100 kD, 50 kD to 90 kD, or 60 kD to 80 kD. Further, for example, the branched PEG has one branch, and the average molecular weight of each PEG chain extending from the branch can be independently, for example, 20 kD to 60 kD, 25 kD to 50 kD, or 30 kD to 40 kD. According to the present invention, such a copolymer of branched PEG and cationic polymer can be administered as the cationic polymer of the present invention.

In a further specific embodiment, the average degree of polymerization of the cationic polymer block is 15 to 30, the cationic polymer block is linked to a branched PEG, and the branched PEG has one branch, and the average molecular weight of each PEG chain extending from the branch can be independently, for example, 20 kD to 60 kD, 25 kD to 50 kD, or 30 kD to 40 kD. According to the present invention, such a copolymer of branched PEG and cationic polymer can be administered as the cationic polymer of the present invention.

In the present invention, Cationic polymer or cationic polymer portion include, for example, cationic natural amino acids and cationic non-natural amino acids, for example, cationic natural amino acids such as histidine, tryptophan, ornithine, arginine, and lysine, and/or a polymer block having a group represented by —(NH—(CH₂)₂)ₚ—NH₂ as a side chain {where p is an integer from 1 to 5}, for example, a polymer block of a cationic non-natural amino acid having the above cationic side chain, for example, a polymer block of a cationic non-natural amino acid such as aspartic acid or glutamic acid having the above cationic side chain. In one embodiment of the present invention, the polycation block is a polymer block having a group represented by —(NH—(CH₂)₂)ₚ—NH₂ as a side chain {where p is an integer from 1 to 5}. Here, Cationic natural amino acids preferably include histidine, tryptophan, ornithine, arginine, and lysine; more preferably arginine, ornithine, and lysine; still more preferably ornithine and lysine; and even more preferably lysine. In one embodiment of the present invention, the cationic polymer or cationic polymer portion can be polylysine or polyornithine.

The polycation block may contain a mixture of cationic amino acids and amino acids having a cationic side chain. That is, in one embodiment of the present invention, the polycation block is a polymer of monomer units comprising cationic natural amino acids, cationic non-natural amino acids, or cationic natural amino acids and cationic non-natural amino acids. In one embodiment of the present invention, the bonds between the monomer units in the polycation block are peptide bonds. In a preferred embodiment of the present invention, the cationic non-natural amino acid is an amino acid having a group represented by —(NH—(CH₂)₂)ₚ—NH₂ as a side chain {where p is an integer from 1 to 5}. In one embodiment of the present invention, the polycation block can be a polycation block formed by polymerization of cationic natural amino acids and aspartic acid and glutamic acid modified with a group represented by —(NH—(CH₂)₂)ₚ—NH₂ {where p is an integer from 1 to 5} in any order. In one embodiment of the present invention, 40%, 50%, 60%, 70%, 80%, 90%, 95%, 98%, or 100% of the monomer units in the polymer have a group represented by —(NH—(CH₂)₂)ₚ—NH₂ as a side chain {where p is an integer from 1 to 5}.

For example, one embodiment of the cationic polymer of the present invention is polylysine or polyornithine having branched PEG, wherein the PEG portion is a branched PEG having two PEG chains with an average molecular weight of 20 to 50 kD, and the polylysine or polyornithine has an average degree of polymerization of 20 to 70, or 30 to 60. In one preferred embodiment, the PEG or branched PEG can be linked to the terminus of the cationic polymer. In another embodiment, the polymer is a polymer with a main backbone of cationic monomers such as lysine and ornithine, wherein 5% to 80%, or 20% to 50% of the side chains of the monomer units are modified with a hydrophilic polymer such as PEG or branched PEG (i.e., a graft copolymer).

In one embodiment, the cationic polymer has a branched PEG at one end of the cationic polymer block. In one embodiment, the cationic polymer has a branched PEG having two PEG chains at one end of the cationic polymer block. In one embodiment, the cationic polymer block can be a homopolymer or a copolymer of lysine or ornithine.

In one embodiment, the cationic polymer can be administered, for example, immediately before administration of the lipid nanoparticle encapsulating the molecule of interest, for example, 30 seconds or more before the administration, 1 minute or more, 2 minutes or more, 3 minutes or more, 4 minutes or more, or 5 minutes or more before the administration. In one embodiment, the cationic polymer can be administered, for example, within 60 minutes, 50 minutes, 40 minutes, 30 minutes, 20 minutes, or 10 minutes before administration of the lipid nanoparticle encapsulating the molecule of interest. In one embodiment, the lipid nanoparticle encapsulating the molecule of interest can be administered 10 minutes before, 5 minutes before, 4 minutes before, 3 minutes before, 2 minutes before, 1 minute before, 30 seconds before, or immediately before administration of the cationic polymer, and it is preferable that the time from molecule of interest administration to cationic polymer administration is short. In the case of simultaneous administration, for example, the lipid nanoparticle encapsulating the molecule of interest and the cationic polymer can be mixed in an infusion bag and administered.

The cationic polymer of the present invention can be administered before, simultaneously with, or after administration of the lipid nanoparticle encapsulating the nucleic acid. In any case, when the cationic polymer and the drug are administered separately, control of pharmacokinetics (particularly, delivery of the lipid nanoparticle to the spleen) is possible when both are simultaneously present in the blood {if the cationic polymer is administered before administration of the lipid nanoparticle encapsulating the molecule of interest, the lipid nanoparticle is administered while the cationic polymer is retained in the blood or remains on the vascular surface of sinusoidal endothelial cells in the liver or remains on the vascular surface of endothelial cells in the kidney; if the cationic polymer is administered after administration of the drug, the cationic polymer is administered while the lipid nanoparticle is retained in the blood}. In one embodiment, the cationic polymer can be administered, for example, immediately before drug administration. For example, it can be administered 30 seconds or more before, 1 minute or more, 2 minutes or more, 3 minutes or more, 4 minutes or more, or 5 minutes or more before the administration. In one embodiment, the cationic polymer can be administered, for example, within 60 minutes, 50 minutes, 40 minutes, 30 minutes, 20 minutes, or 10 minutes before administration of the lipid nanoparticle. In one embodiment, the lipid nanoparticle can be administered 10 minutes before, 5 minutes before, 4 minutes before, 3 minutes before, 2 minutes before, 1 minute before, 30 seconds before, or immediately before administration of the cationic polymer, and it is preferable that the time from lipid nanoparticle administration to cationic polymer administration is short. In the case of simultaneous administration, for example, the lipid nanoparticle and the cationic polymer can be mixed in an infusion bag and administered.

The lipid nanoparticle of the present disclosure is not a naturally occurring particle. The lipid nanoparticle of the present disclosure may have a viral genome in one embodiment, but does not have a viral genome in another embodiment.

The lipid nanoparticle of the present disclosure preferably comprises an ionizable lipid in one preferred embodiment. That is, the lipid nanoparticle of the present disclosure can be an ionizable lipid-based nanoparticle (iLNP). The lipid nanoparticle of the present disclosure is preferably prepared by a microfluidic device in one preferred embodiment. In one embodiment, the lipid nanoparticle of the present disclosure preferably comprises an ionizable lipid and is constructed using a microfluidic device to encapsulate the molecule of interest inside the nanovesicle. The lipid nanoparticle of the present disclosure may have a positive charge in a neutral aqueous solution.

In the method of the present disclosure, an effective amount of the cationic polymer may be administered, but it may be administered at a dose that does not cause dose-limiting toxicity (DLT).

According to the present disclosure, the lipid nanovesicle can selectively accumulate in the spleen when used in combination (co-used) with the cationic polymer. Therefore, it is preferable that the lipid nanoparticle encapsulates a molecule that provides a physiological benefit (including a medical benefit) by selectively accumulating in the spleen. The molecule that provides a physiological benefit (including a medical benefit) by selectively accumulating in the spleen can be, for example, an immunogen (also called an antigen) or a nucleic acid encoding an immunogen. Further, in one preferred embodiment, when the lipid nanovesicle is used in combination (co-used) with the cationic polymer, the distribution to organs other than the liver and spleen (e.g., the heart) does not increase compared to the case where it is not co-used.

An immunogen is a molecule (typically a protein or peptide) that can induce a specific immune response in the body of an individual. An immunogen is recognized and processed by the immune system (e.g., dendritic cells or macrophages), becomes a fragment of the immunogen, and is presented to other immune cells (e.g., T cells and B cells). An immune response can induce immunogen-specific cellular immunity and/or immunogen-specific humoral immunity (e.g., antibodies). T cells and B cells are produced in the thymus and bone marrow, respectively, and migrate to secondary lymphoid tissues such as the spleen, where they induce an immune response. Therefore, the delivery of the molecule of interest to secondary lymphoid tissues is suitable for, for example, inducing immunogen-specific immunity. Alternatively, the delivery of the molecule of interest to secondary lymphoid tissues is also suitable for, for example, inducing immune activation (non-specific activation).

Examples of immunogens include viral antigens, bacterial antigens, fungal antigens, parasite antigens, and tumor antigens. Examples of viral antigens include influenza virus antigens such as hemagglutinin (HA) and neuraminidase (NA); human immunodeficiency virus (HIV)-1 or HIV-2 antigens such as gp120, gp160, and p24; hepatitis B virus (HBV) antigens such as HBV surface antigen (HBsAg) and HBV core antigen (HBVcAg); hepatitis C virus (HCV) antigens such as HCV core antigen, E1, and E2; coronavirus antigens (e.g., β coronavirus, e.g., SARS-COV-2 (e.g., α strain, β strain, γ strain, δ strain, and o strain, e.g., JN.1, XBB.1.5, XBB.1.16, EG.5, BA.2.86, XBB, XBB.1.9.1, XBB.2, and other variant strains)) such as spike(S) protein, nucleocapsid (N) protein, and envelope (E) protein; human papillomavirus (HPV) antigens such as L1 capsid protein and L2 capsid protein; and rotavirus antigens such as VP4 and VP7. Examples of bacterial antigens include Streptococcus pneumoniae antigens (such as PspA, PspC), Neisseria meningitidis antigens (such as PorA, PorB), Mycobacterium tuberculosis antigens (such as Ag85B, ESAT-6), Haemophilus influenzae type e antigen (such as PRP), Bordetella pertussis antigens (such as PT, FHA, PRN), Clostridium tetani antigen (such as TeNT), and Clostridium difficile antigens (such as A and B toxins). Furthermore, examples of fungal antigens include Candida albicans antigens (such as Als3, Hwp1), Aspergillus fumigatus antigens (such as Asp f1, Asp f2), Paracoccidioides brasiliensis antigens (such as Gp43), Coccidioides antigens (such as Ag2/PRA obtained from Coccidioides posadasii and Coccidioides immitis), Cryptomeria japonica antigens (such as Cry j1, Cry j2), and Candida glabrata antigens (such as Eno1, Fks1). Furthermore, examples of parasite antigens include Plasmodium falciparum antigens (such as CSP, MSP1), Toxoplasma gondii antigens (such as SAG1, GRA1), Entamoeba histolytica antigens (such as Gal/GalNAc), Leishmania antigens (such as GP63, LPG), Plasmodium falciparum antigens (such as VAR2CSA, PfEMP1), Plasmodium vivax antigens (such as LDH, AMA1), Schistosoma mansoni antigens (such as Sm28GST, Sm23), Trichinella spiralis antigens (such as TES-120, TES-30), Onchocerca volvulus antigens (such as Ov-16, Ov-39), and Leishmania donovani antigens (such as rK39, rK28). Furthermore, examples of tumor antigens include any protein whose expression is observed in cancer cells, for example, Wilms Tumor 1 (WT-1), Human Carbohydrate Antigen 125 (CA-125), Carcinoembryonic Antigen (CEA), Human Telomerase Reverse Transcriptase (hTERT), Mucin-1 (Muc-1), Mucin-2 (Muc-2), Cancer/Testis antigen 1B (CTAG1B/NY-ESO-1), Prostatic Acid Phosphatase (PAP), Prostate Specific Antigen (PSA), Prostate Specific Membrane Antigen (PSMA), Survivin b, mutated ras, mutated p53, and the like. In the present disclosure, a nucleic acid, particularly mRNA, can encode these immunogens or a part thereof (e.g., having an amino acid length of 9 or more, 10 or more, 11 or more, 12 or more, 13 or more, 14 or more, or 15 or more).

In one embodiment, the lipid nanoparticle comprises an ionizable lipid, a non-cationic lipid (preferably neutral lipid), cholesterol, and a PEG lipid, and the molecule of interest is mRNA encoding any one or more of the above immunogens.

In one embodiment, the method of the present disclosure can prevent the lipid nanoparticle from migrating to the liver in the subject by administering the cationic lipid. In one embodiment, the method of the present disclosure can prevent immune tolerance to the antigen, which may occur due to antigen expression in the liver, by administering the cationic lipid.

In one embodiment, the method of the present disclosure induces immunogen-specific T cell immunity in the subject. In one embodiment, the method of the present disclosure induces immunogen-specific antibodies in the subject.

In one embodiment, the method of the present disclosure comprises intravenously administering the lipid nanovesicle. In one embodiment, the method of the present disclosure comprises subcutaneously, intradermally, or intramuscularly administering the lipid nanovesicle.

In one embodiment, the mRNA has a target sequence for microRNA within its UTR. The target sequence for microRNA has a sequence complementary to the microRNA. When the mRNA has a target sequence for microRNA, the expression of the protein from the mRNA is suppressed in the presence of the microRNA. For example, by using a microRNA that is expressed in the liver, the expression of the protein from the mRNA can be suppressed in the liver. Examples of microRNAs expressed in the liver include miR-122, miR-192, miR-34a, miR-33a/b, miR-29, miR-221/222, miR-155, miR-199a/b, and miR-27a/b, and these are preferably human microRNAs, respectively. In one preferred embodiment, the microRNA is miR-122, and the target sequence is a sequence complementary to miR-122, for example, the RNA sequence encoded by the DNA described in SEQ ID NO: 2.

The mRNA may have multiple target sequences for microRNA within its UTR. The multiple target sequences may be the same or different, and may include 2 to 5 identical target sequences. If the target sequences are different, they may include target sequences for different microRNAs.

In one embodiment, the mRNA may include one or a plurality (e.g., 2 to 5) of target sequences for miR-122 within its 3′ UTR.

In one embodiment,
the lipid nanoparticle comprises an ionizable lipid, a non-cationic lipid (preferably neutral lipid), cholesterol, and a PEG lipid, the molecule of interest is mRNA encoding any one or more of the above immunogens, and
the cationic polymer is a block copolymer with a non-charged hydrophilic polymer.

In one embodiment,
the lipid nanoparticle comprises an ionizable lipid, a non-cationic lipid (preferably neutral lipid), cholesterol, and a PEG lipid, the molecule of interest is mRNA encoding any one or more of the above immunogens, and
the cationic polymer is a block copolymer with polyethylene glycol.

In one embodiment,
the lipid nanoparticle comprises an ionizable lipid, a non-cationic lipid (preferably neutral lipid), cholesterol, and a PEG lipid, the molecule of interest is mRNA encoding any one or more of the above immunogens, and
the cationic polymer is a block copolymer with a branched polyethylene glycol chain.

In one embodiment,
the lipid nanoparticle comprises an ionizable lipid, a non-cationic lipid (preferably neutral lipid), cholesterol, and a PEG lipid, the molecule of interest is mRNA encoding any one or more of the above immunogens, and
the cationic polymer is a block copolymer of a homopolymer or copolymer of a cationic amino acid selected from lysine and ornithine, and a branched polyethylene glycol chain. Here, the branched polyethylene glycol may preferably have two polyethylene glycol chains.

According to the present disclosure, a composition comprising the cationic polymer for use in the method of the present disclosure is provided. According to the present disclosure, a composition comprising the lipid nanoparticle for use in the method of the present disclosure is provided. According to the present disclosure, a composition comprising the cationic polymer, a composition comprising the lipid nanoparticle, and a combination product of the compositions, for use in the method of the present disclosure, are provided. According to the present disclosure, a composition comprising the cationic polymer and the lipid nanoparticle for use in the method of the present disclosure is provided.

According to the present disclosure, use of a cationic polymer in the manufacture of a medicament for use in the method of the present disclosure is provided. According to the present disclosure, use of a lipid nanoparticle in the manufacture of a medicament for use in the method of the present disclosure is provided. According to the present disclosure, use of a cationic polymer and a lipid nanoparticle in the manufacture of a medicament for use in the method of the present disclosure is provided. According to the present disclosure, use of a composition comprising a cationic polymer and a lipid nanoparticle in the manufacture of a medicament for use in the method of the present disclosure is provided. According to the present disclosure, use of a combination of a cationic polymer and a lipid nanoparticle in the manufacture of a medicament for use in the method of the present disclosure is provided.

In one embodiment, the subject has a liver disorder or disease. According to the method of the present disclosure, the burden on the liver in the subject can be reduced by the reduced delivery of the molecule of interest to the liver. Examples of liver disorders or diseases include liver dysfunction and liver diseases such as hepatitis, liver fibrosis, cirrhosis, fatty liver (non-alcoholic fatty liver disease (NAFLD) or metabolic dysfunction-associated fatty liver disease (MAFLD)), and liver cancer.

### EXAMPLES

### Example 1

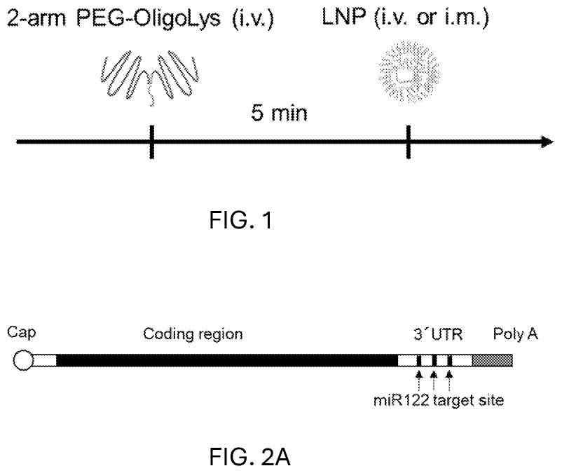

Administration of the molecule of interest to the subject was attempted with a scheme in which lipid nanoparticles (LNP) containing the molecule of interest were administered to a subject who had been administered a cationic polymer (e.g., a block copolymer of a hydrophilic polymer and a cationic polymer) (see, e.g., FIG. 1).

The block copolymer was one comprising polyethylene glycol as the hydrophilic polymer and poly-L-lysine as the cationic polymer, more specifically, 2-arm-PEG-PLys, which has two PEG chains with a number average molecular weight of 40k bonded to the terminus of an oligolysine with a number average degree of polymerization of 20 (see Science Advances 6, eabb8133, (2020)).

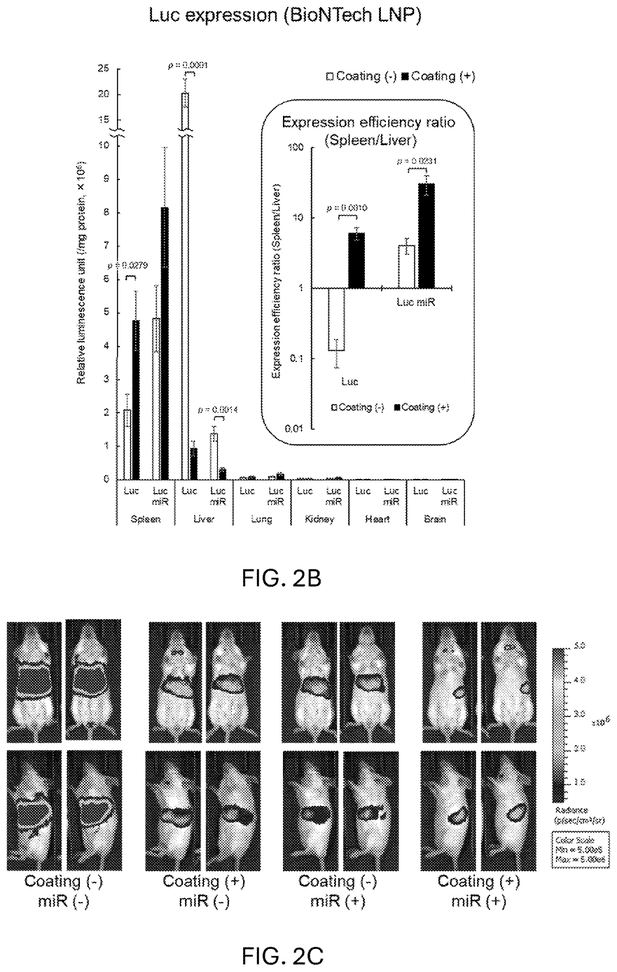

The LNP was prepared as follows. A lipid solution was obtained by dissolving ionizable lipid (ALC-0315, MedChemExpress, Monmouth Junction, NJ, USA), phospholipid (1,2-distearoyl-sn-glycero-3-phosphocholine, DSPC, Fujifilm Wako, Osaka, Japan), cholesterol (Sigma Aldrich), and PEG lipid (ALC-0159, MedChemExpress) in an ethanol solvent at a molar ratio of 46.3:9.4:42.7:1.6. An mRNA solution containing mRNA dissolved in a pH 3 citrate buffer was obtained. The lipid solution and the mRNA solution were mixed in a microfluidic channel (NanoAssemblr (trademark) Spark, Precision Nanosystems, Vancouver, Canada) at a volume ratio of 2:1 and a molar ratio of the amino group (N) in ALC-315 to the phosphate group (P) in mRNA (N/P ratio) of 6 to obtain lipid nanoparticles encapsulating the mRNA. The resulting lipid nanoparticles were diluted 40-fold with PBS and then concentrated using Amicon Ultra-15-30K centrifugal units (Merck Millipore).

Where specified, the following sequences (control and miR 122 target sequence) were inserted downstream of the Luc2 sequence (Promega) in the mRNA (see FIG. 2A). This suppressed protein synthesis from the mRNA delivered to the liver. The resulting mRNAs are referred to as miR(−) and miR(+), respectively.

Cont (SEQ ID NO: 1):
TGATAATAGGCTGGAGCCTCGGTGGCCATGCTTCTTGCCCCTTGGGCCT
CCCCCCAGCCCCTCCTCCCCTTCCTGCACCCGTACCCCCGTGGTCTTTG
AATAAAGTCTGAGTGGGCGGC
miR (SEQ ID NO: 2):
TGATAATAGCAAACACCATTGTCACACTCCAGCTGGAGCCTCGGTGGCC
ATGCTTCTTGCCCCTTGGGCCCAAACACCATTGTCACACTCCATCCCCC
CAGCCCCTCCTCCCCTTCCTGCACCCGTACCCCCCAAACACCATTGTCA
CACTCCAGTGGTCTTTGAATAAAGTCTGAGTGGGCGGC

A plasmid having the DNA encoding the above mRNA was amplified by PCR using a primer containing an 80-base poly T sequence, and mRNA having an 80-base poly A chain was prepared using the mMessage mMachine T7 kit (Thermofisher, Waltham, MA).

Balb/c 7-week-old female mice were purchased and used. 1.25 mg of 2-arm-PEG-PLys was administered via the tail vein, and 5 μg of LNP loaded with mRNA was administered via the tail vein 5 minutes later. Luciferase expression was analyzed 4 hours later. The cationic polymer is thought to coat the surface of the sinusoidal endothelial cells in the liver and inhibit the liver's metabolism of the active ingredient. Therefore, the cationic polymer administration group is sometimes referred to as Coating (+).

The organs were homogenized in passive lysis buffer (Promega) with a bead-based cell disruptor (Tommy Seiko MS-100). The homogenate was mixed with the Luciferase assay system (Promega), and the luminescence was measured with a Lumat3 LB9508 luminometer (Berthold technologies, Bad Wildbad, Germany). The protein amount was measured using the Micro BCA Protein Assay Kit (ThermoFisher), and the luminescence was corrected. The results are shown in FIG. 2B. In FIG. 2B, the inserted graph shows the value of the spleen divided by the value of the liver for each mouse (n=5). As shown in FIG. 2B, high mRNA expression was observed in the spleen with 2-arm-PEG-PLys. Also, reduced mRNA expression was observed in the liver with 2-arm-PEG-PLys. The increase in mRNA expression in the spleen was considered to be due to the pre-administration of 2-arm-PEG-PLys, and the decrease in mRNA expression in the liver was considered to be due to the introduction of the miR122 target sequence into the UTR of the mRNA.

Further, 3 mg of luciferin (Promega) was administered intraperitoneally to the mice treated as described above, and the distribution of Luc2 expression in the mice was observed with the IVIS Spectrum imaging system (SP-BFM-T1, PerkinElmer, Waltham, MA, USA). The results are shown in FIG. 2C. As shown in FIG. 2C, mRNA expression was confirmed in the liver and spleen in the cationic polymer administration group, while mRNA expression in the liver was significantly suppressed. Furthermore, when miR(+) was administered to the cationic polymer administration group, it was shown that the mRNA was selectively expressed in the spleen. Since mRNA expression in the liver was higher than in the spleen when miR(+) was administered to the non-cationic polymer administration group, the co-administration of the cationic polymer was considered to have the effect of suppressing LNP metabolism in the liver and increasing mRNA expression in the spleen.

### Example 2

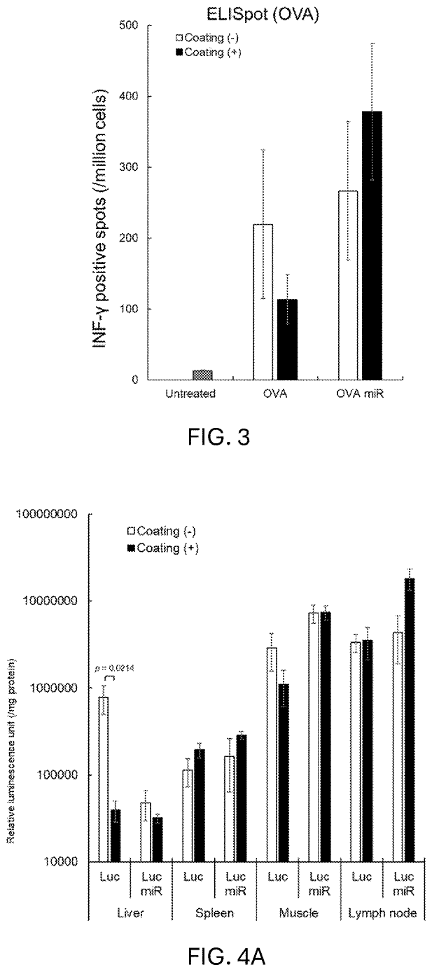

Next, the induction of antigen-specific immunity was attempted using the administration scheme shown in FIG. 1. The mRNA encoded ovalbumin. mRNA was prepared containing the control or the miR 122 target sequence as shown in FIG. 2A. 1.25 mg of 2-arm-PEG-PLys was administered via the tail vein to purchased mice (C57BL6J, 7-week-old female), and 5 μg of LNP loaded with mRNA was administered via the tail vein 5 minutes later. This administration was considered one set, and another set was added 2 weeks after the first set. Splenocytes were collected 1 week after the second set. The splenocytes at 2.5×10⁵ cells/well were plated in an anti-IFNγ-ELISpot 96 well plate (Mabtech, Nacka Strand, Sweden), and PepTivator Ovalbumin epitope mix (Miltenyi Biotec (Nordrhein-Westfalen, Germany)) was added at 0.025 μg/well. After culturing overnight at 37° C. and 5% CO₂, the cells were processed according to the kit protocol, and the number of spots was observed with an ELISpot plate reader (AID GmbH, Germany) (N=6). The results are shown in FIG. 3. As shown in FIG. 3, induction of antigen-specific immune cells was confirmed.

### Example 3

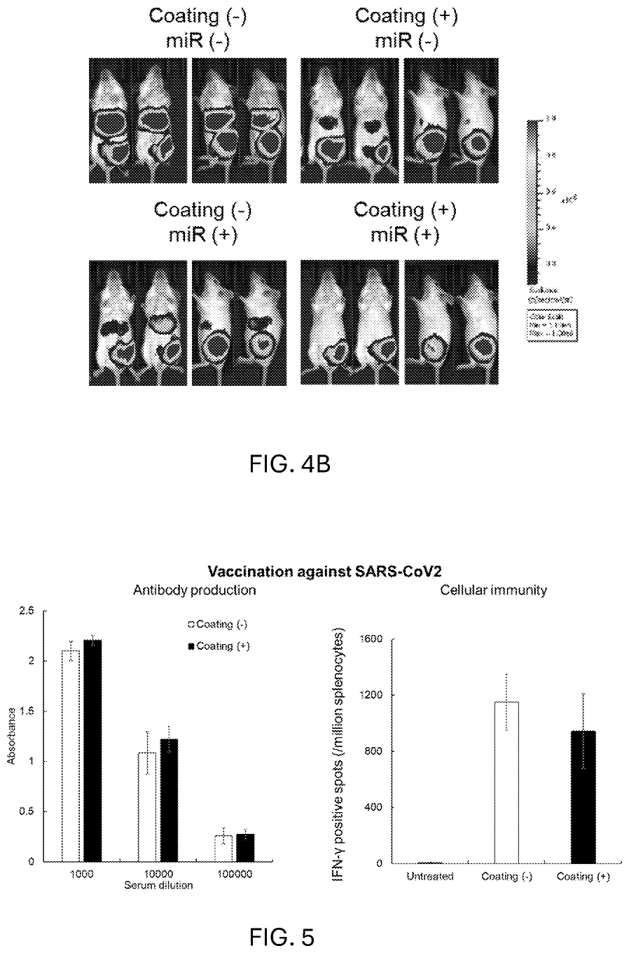

The cationic polymer and mRNA were prepared as in Example 1. 1.25 mg of 2-arm-PEG-PLys was administered via the tail vein to purchased Balb/c 7-week-old female mice, and 5 μg of LNP loaded with mRNA was administered intramuscularly 5 minutes later. Luciferase expression was measured 4 hours later. Each organ was harvested and homogenized in the same manner as in Example 1, and the luciferase expression level in the homogenate was measured. The results are shown in FIG. 4A. As shown in FIG. 4A, luciferase expression in the liver decreased. Thus, in the cationic polymer administration group of the present disclosure, expression in the spleen and lymph nodes did not decrease (rather, an increasing trend was observed), and expression in the liver was significantly decreased. In vivo fluorescence imaging was performed as in Example 1. The results are shown in FIG. 4B. As shown in FIG. 4B, spleen-selective luciferase expression was observed when miR(+) was administered in the cationic polymer administration group.

### Example 4

The induction of specific immunity against SARS-COV-2 was attempted. mRNA expressing the Wuhan-type spike protein (with N1mψ modification) was purchased from Trilink. The miR 122 target sequence was introduced into the 3′ UTR as shown in FIG. 2A. 1.25 mg of 2-arm-PEG-PLys was administered via the tail vein, and 5 μg of LNP solution containing mRNA was administered into the thigh muscle 5 minutes later. The administration was performed twice every 3 weeks, and the antibody amount was measured by ELISA from the collected blood sample 2 weeks after the second administration, and cellular immunity was quantified by ELISpot from the spleen.

The antibody amount was measured as follows. Recombinant spike protein (Sino Biological) was dissolved in carbonate buffer (50 mM, pH=9.6) at 2 μg/mL, and 50 μL/well was added to Clear Flat-Bottom Immuno Nonsterile 96-Well Plates (Thermo). After standing overnight at 4° C., the plates were washed 3 times with 0.5% v/v Tween 20 in PBS (PBS-T). 100 μL of 1% BSA and 2.5 mM EDTA in PBS-T was added, and the plates were allowed to stand for 1 hour at room temperature. 50 μL of plasma was added, and the plates were allowed to stand overnight at 4° C. The plates were washed 3 times with PBS-T, and 50 μL of goat anti-mouse IgG (1:8000) was added, and the plates were allowed to stand for 2 hours at room temperature. 100 μL/well of HRP substrate was added, and the plates were allowed to stand for 30 minutes at room temperature. The reaction was stopped with 2M sulfuric acid, and the absorbance at 492 nm was measured with a plate reader (Tecan, Switzerland). ELISpot was performed in the same manner as in Example 2, using an anti-IFNγ-ELISpot 96 well plate (Mabtech) and 0.2 μg/well of PepMix™ SARS-COV-2 (S) (JPT Peptide technologies, Berlin, Germany) (N=4).

The results are shown in FIG. 5. As shown in FIG. 5, antibody production was higher in the cationic polymer administration group than in the non-administration group. No statistically significant difference in cellular immunity was observed between the cationic polymer administration group and the non-administration group.

### Example 5

MC3 LNP was prepared by mixing ionizable lipid (D-Lin-MC3-DMA; (6Z,9Z,28Z,31Z)-heptatriaconta-6,9,28,31-tetraen-19-yl-4-(dimethylamino)butanoate), 1,2-distearoyl-sn-glycero-3-phosphocholine (DSPC), cholesterol, and PEG lipid (PEG2000-dimethylglycerol (DMG)) (50:10:38.5:1.5 mol %, ethanol solvent) with Luc2 mRNA (see Example 1) in a pH 3 citrate buffer in a microfluidic channel (NanoAssemblr® Spark, Precision Nanosystems, Vancouver, Canada) at a volume ratio of 2:1 and a molar ratio of the amino group (N) in D-Lin-MC3-DMA to the phosphate group (P) in mRNA (N/P ratio) of 5. The resulting LNP was diluted 40-fold with PBS and then concentrated using Amicon Ultra-15-30K centrifugal units (Merck Millipore).

Balb/c 7-week-old female mice were purchased and used. 1.25 mg of 2-arm-PEG-PLys was administered via the tail vein, and 5 μg of LNP loaded with mRNA (LNP prepared in Example 4) was administered via the tail vein 5 minutes later. Luciferase expression was analyzed 4 hours later. The organs were homogenized in passive lysis buffer (Promega) with a bead-based cell disruptor (Tommy Seiko MS-100). The homogenate was mixed with the Luciferase assay system (Promega). Then, the luminescence was measured with a Lumat3 LB9508 luminometer (Berthold technologies, Bad Wildbad, Germany) (N=3).

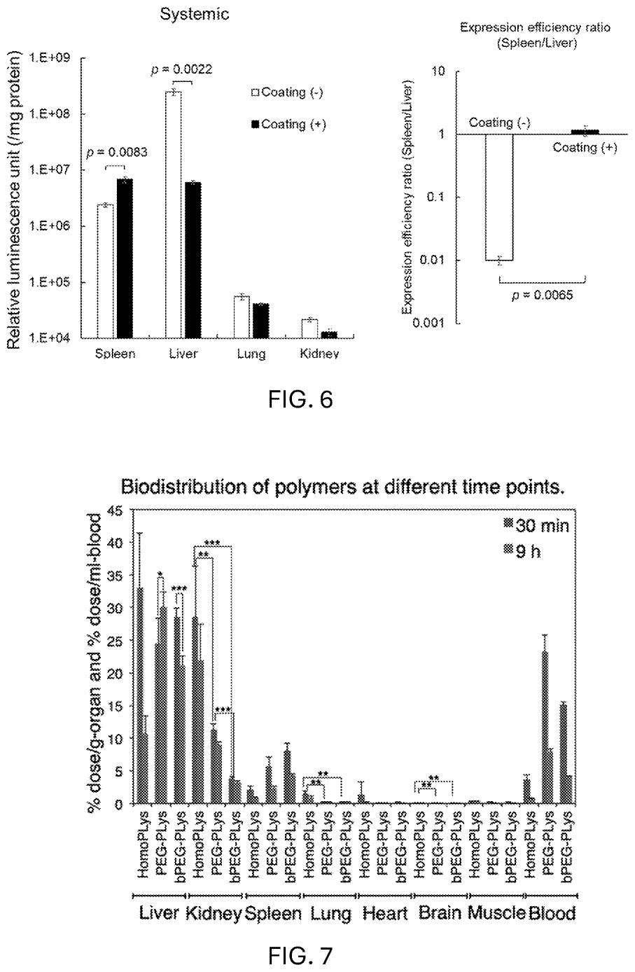

As a result, as shown in FIG. 6, mRNA expression levels in the liver and kidney decreased, and mRNA expression in the spleen increased.

### Example 6

0.05 mg of Cy5-labeled OligoLys 20-mer (HomoPLys), 1.25 mg of OligoLys 20-mer bonded with one 80 kDa PEG (PEG-PLys), and 1.25 mg of OligoLys 20-mer bonded with two 40 kDa PEG chains (bPEG-PLys) were administered intravenously to mice, and the distribution of the administered cationic polymers in organs was observed. Balb/c 7-week-old female mice were purchased and used. The organs were homogenized in Passive Lysis buffer, and the fluorescence was measured with a plate reader (Tecan). As shown in FIG. 7, even in the bPEG-PLys administration group, the accumulation of the polymer was observed to have high selectivity for the liver. In contrast, in the homo-pLys administration group, PEG-pLys administration group, and bPEG-PLys administration group, the amount of polymer accumulation selectively increased in the spleen. Furthermore, no increase in the accumulation of the cationic polymer in organs other than the spleen was observed. This result indicates that the cationic polymer showed high hepatic tropism while exhibiting preferential accumulation in the spleen. As mentioned above, since the presence of the cationic polymer reduces LNP distribution to the liver and enhances selective splenic delivery, it is highly probable that the cationic polymer preferentially accumulates in the liver in vivo and blocks LNP distribution to the liver. When LNP was administered in combination with the cationic polymer, no increase in LNP accumulation in organs other than the spleen was observed. This result was surprising in that it was a contrasting result to US2021/0038634A, where the co-administration of a cationic polymer enhanced the delivery efficiency of polyplex micelles and AAV8 to the myocardium and skeletal muscle. Since the co-administration of the cationic polymer and LNP does not increase delivery efficiency to the myocardium, it was suggested that the cationic polymer does not have an adverse effect on myocarditis that may be caused by LNP.

### Example 7

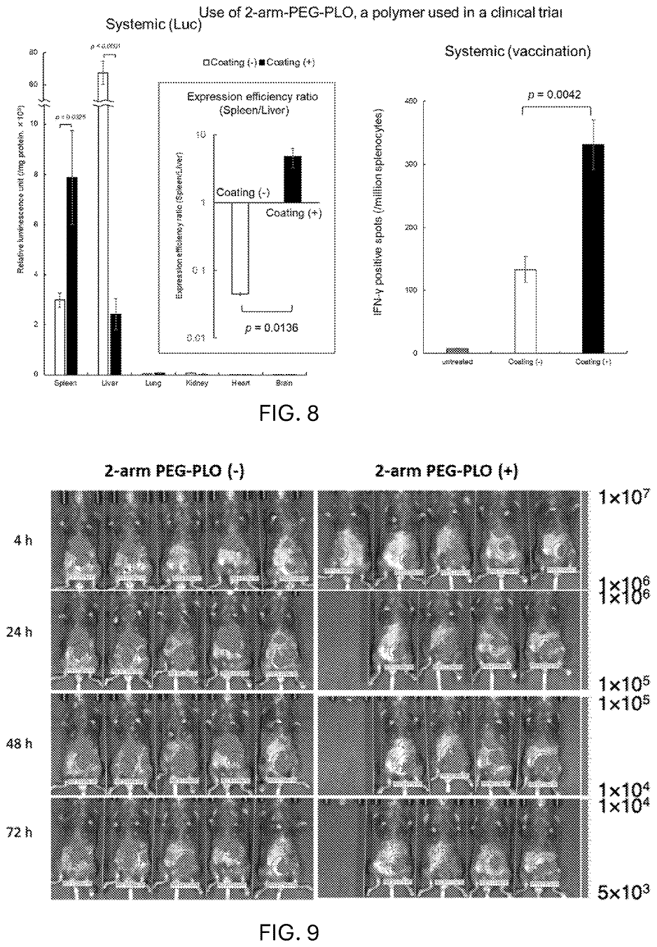

Balb/c 7-week-old female mice were purchased and used. 1.25 mg of Oligo-L-ornithine bonded with two 40 kDa PEG chains (2-arm-PEG-PLO) was administered intravenously, and 5 μg of Luc2 cont mRNA was administered intravenously 5 minutes later using the same LNP as in Example 1. The degree of polymerization of the Oligo-L-ornithine was 20. Luciferase was measured 4 hours later. The organs were homogenized in passive lysis buffer (Promega) with a bead-based cell disruptor (Tommy Seiko MS-100). The homogenate was mixed with the Luciferase assay system (Promega), and the luminescence was measured with a Lumat3 LB9508 luminometer (Berthold technologies, Bad Wildbad, Germany). The protein amount was measured using the Micro BCA Protein Assay Kit (ThermoFisher), and the luminescence was corrected. The results are shown in FIG. 8. The graph enclosed in a square in FIG. 8 shows the value of the spleen divided by the value of the liver for each mouse (n=5). As shown in FIG. 8, the mRNA expression level in the spleen increased and the expression level in the liver decreased in the cationic polymer administration group.

C57BL6J 7-week-old female mice were purchased and used. A vaccine experiment was performed using sOVA cont mRNA (without the miR target sequence) from Example 2. LNP was prepared in the same manner as in Example 1. 1.25 mg of 2-arm-PEG-PLys was administered via the tail vein, and 5 μg of LNP loaded with mRNA was administered via the tail vein 5 minutes later. This administration was considered one set, and another set was added 2 weeks after the first set. Splenocytes were collected 1 week after the second set. The splenocytes (2.5×10⁵ cells/well) were plated in an anti-IFNγ-ELISpot 96 well plate (Mabtech, Nacka Strand, Sweden). PepTivator Ovalbumin epitope mix (Miltenyi Biotec (Nordrhein-westfalen, Germany) was added at 0.025 μg/well to OVA epitope mix. After culturing overnight at 37° C. and 5% CO₂, the cells were processed according to the kit protocol, and the number of spots was observed with an ELISpot plate reader (AID GmbH, Germany) (N=4). As a result, as shown in FIG. 8, induction of OVA-specific cellular immunity was confirmed in the ELISpot assay. It is considered that the enhanced mRNA expression in the spleen when the cationic polymer was co-administered led to an enhancement of the vaccine effect.

### Example 8

B16F10 cells were subcutaneously grafted into C57BL6J mice. 1.25 mg of 2-arm-PEG-PLO was administered intravenously, or nothing was administered. 5 μg of Luc2 cont mRNA (without the miR target sequence) was administered directly into the tumor 5 minutes later using the same LNP as in Example 1. After a certain period of time, 3 mg of luciferin (Promega) was administered intraperitoneally, and the IVIS Spectrum imaging system (SP-BFM-T1, PerkinElmer, Waltham, MA, USA) was used for observation. The results are shown in FIG. 9. As shown in FIG. 9, mRNA was selectively expressed in the spleen in the 2-arm-PEG-PLO administration group.

### Example 9

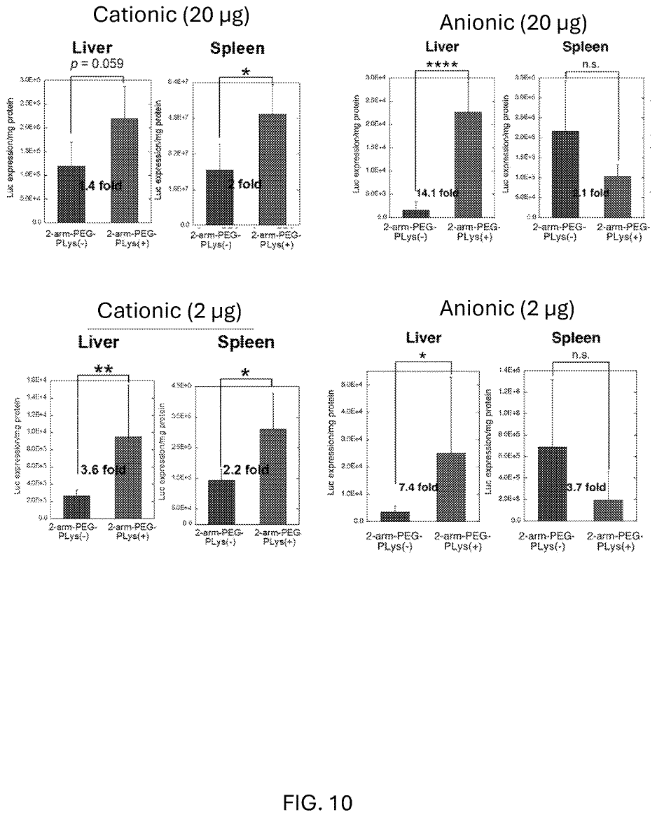

A liposome was prepared by a thin film method by mixing the cationic lipid 1,2-di-O-octadecenyl-3-trimethylammonium propane (DOTMA) and the phospholipid 1,2-dioleoyl-sn-glycero-3-phosphoethanolamine (DOPE) at a molar ratio of 1:1. An anionic lipoplex was prepared by adding mRNA such that the molar ratio of the amino group (N) in DOTMA to the phosphate group (P) in mRNA (N/P) was 0.5. A cationic lipoplex was prepared by adding mRNA such that the N/P ratio was 5. mRNA expressing Luc2 was used. Balb/c 7-week-old female mice were purchased and used. 1.25 mg of 2-arm-PEG-PLys was administered intravenously, or nothing was administered, and the lipoplex was administered intravenously 5 minutes later. The spleen and liver were harvested 4 hours later. The organs were homogenized in the same manner as in Example 1, and the luciferase expression was quantified. The results were as shown in FIG. 10. As shown in FIG. 10, in the cationic polymer administration group, the anionic lipoplex increased the mRNA expression level in the liver and decreased the mRNA expression in the spleen compared to the non-administration group. In contrast, in the cationic polymer administration group, the cationic lipoplex increased the mRNA expression in the spleen compared to the non-administration group.

1. A method of delivering a molecule of interest to the spleen of a subject in need thereof, comprising administering to the subject a cationic polymer and a lipid nanoparticle (LNP) encapsulating the molecule of interest, wherein at least a portion of the cationic polymer is administered simultaneously with the LNP administration or before the LNP administration.

2. The method according to claim 1, wherein the cationic polymer is a block copolymer with a non-charged hydrophilic polymer.

3. The method according to claim 1 or 2, wherein the LNP comprises (i) a cationic lipid or ionizable lipid, (ii) a neutral lipid, (iii) cholesterol, and (iv) a PEG lipid.

4. The method according to any one of claims 1 to 3, wherein the molecule of interest is a nucleic acid.

5. The method according to any one of claims 1 to 4, wherein the molecule of interest is a messenger RNA (mRNA).

6. The method according to claim 5, wherein the molecule of interest is an mRNA encoding an antigen protein, thereby causing the antigen protein to be expressed in the spleen of the subject and inducing antigen-specific immunity in the subject.

7. The method according to claim 6, wherein the antigen protein is the spike protein of SARS-COV-2.

8. A composition comprising a cationic polymer for use in the method according to any one of claims 1 to 7.

9. The composition according to claim 8, further for use in suppressing the distribution of the molecule of interest to the liver.

10. The composition according to claim 9, which is a pharmaceutical composition.


SEQUENCE LISTING (2 sequences)

SEQ ID NO: 1 - DNA, 119 bp, synthetic construct
  source 1..119: mol_type = other DNA
tgataataggctggagcctcggtggccatgcttcttgccccttgggcctccccccagcccctcctccccttcctgcacccgtacccccgtggtctttgaataaagtctgagtgggcggc

SEQ ID NO: 2 - DNA, 185 bp, synthetic construct
  source 1..185: mol_type = other DNA
tgataatagcaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggc